(19) United States
(12) Patent Application Publication
(10) Pub. No.: US 2022/0322914 A1
(43) Pub. Date: Oct. 13, 2022

(54) An external endoluminal fixator device

(71) Applicants: Abhijit Chandra, Lucknow (IN); Ravita Srivastava, Lucknow (IN)

(72) Inventors: Abhijit Chandra, Lucknow (IN); Ravita Srivastava, Lucknow (IN); Animesh Chandra, Lucknow (IN)

(21) Appl. No.: 17/761,658

(22) PCT Filed: Sep. 9, 2020
(86) PCT No.: PCT/IB2020/058360

§ 371 Date: Mar. 18, 2022

(30) Foreign Application Priority Data
Sep. 19, 2019 (IN) 201911011061

(51) Int. Cl. A61B 1/005; A61B 1/05; A61B 1/303; A61B 1/31; A61B 17/04; A61B 1/06

(52) U.S. Cl. CPC A61B 1/0055; A61B 1/05; A61B 1/303; A61B 1/31; A61B 17/0469; A61B 1/0669; A61B 2017/00305

(57) ABSTRACT

An external endoluminal fixator device to fix rectal prolapse and/or vaginal and/or uterine and/or urinary bladder prolapse by passing through it, includes a head configured at distal end of the external endoluminal fixator device such that the head passes through an anorectum or a vagina of a body. The device then includes a hollow shaft connected with the head which is in angulation corresponding to usage in the anorectum or a sacral hollow or the vagina of the body and an endoluminal fixator device handle connected with the hollow shaft at proximal end of the external endoluminal fixator device, configured to remain outside of anus or vagina to have a sturdy grip so as to push up the head of the endoluminal fixator device either trans-anally or vaginally with the anorectum or the vagina against undersurface of the anterior abdominal wall.

### FIELD OF THE INVENTION

This invention relates, generally, to a device for surgery and more particularly, to the field of rectal or vaginal/uterine (pelvic organ) prolapse fixation.

### BACKGROUND OF THE INVENTION

Prolapse is a condition in which a body part protrudes from its normal position. Rectal prolapse occurs when the lower most (distal) part of a large bowel “the rectum” protrudes towards or outside the anus. Rectal prolapse is classified into two types: complete (total) and incomplete prolapse (internal). In complete prolapse, referred to as procedentia the entire layer of rectum protrudes to the outside of the anus and in incomplete prolapse or partial, the protrusion of the rectal wall is not full thickness or is limited to the anal canal. Rectal prolapse is often associated with comorbidities like rectal bleed, mucus discharge, incontinence and difficult evacuation. Living with rectal prolapse not only causes embarrassment, but also impairs the quality of life. Surgery is the only definitive method to fix or treat rectal prolapse. More often than not in 20 to 30% of cases rectal prolapse is associated with concomitant prolapse of other compartments of pelvis anterior (urinary bladder-cystocoel) and or middle compartment of vagina/uterus often collectively referred as “Pelvic organ prolapse syndrome” The purpose of surgery is to prevent prolapse, of rectum and or other pelvic structures, restore normal pelvic functions including urination, defecation, constipation and sexual functions To achieve this, dissection of Rectum, vagina, urinary bladder, their suspension and fixation is required, and for this, surgery is performed either through abdomen or the perineum.

More than 100 different procedures for surgical repair of rectal prolapse including other pelvic organs either through perineum (anus or vagina) or abdominal cavity are known and performed.

The perineal approach is less morbid and a conventional technique used to treat rectal prolapse in patients with high operative risk, especially for elderly patients, although with higher recurrence. The abdominal approach is a more aggressive and radical procedure, it is an open surgery that involves extensive bowel dissection/resection and fixation either through laparotomy which is a surgical procedure that involves large incision in the abdomen or laparotomy. Recently abdominal surgery is performed in a minimal invasive manner by small abdominal incisions either with an instrument called a laparoscope or robotically, which are inserted into the abdomen by the surgeon This also requires use of an artificial mesh to fix the prolapsing rectum or vagina or the uterine cervix to the sacral bone usually. Often times another approach called “perinaeal repair” is used for elderly patients and patients with medical conditions. Rectal repair involves fixing the inner lining of the rectum or resecting the portion of the rectum extending out through the anus. Common technique that involves rectal prolapse repair through the area around the anus (perinealrectosigmoidectomy) requires the surgeon to pull the rectum out through the anus, remove a portion of the rectum and sigmoid colon (lower third part of large intestine) and attach the remaining rectum to the large intestine (colon). This repair is typically reserved for those who are not suitable for abdominal (open, laparoscopic or robotic) surgery. Similarly the prolapsing vagina/uterus or urinary bladder can be fixed trans-vaginally or through the perineum simultaneously.

All these procedures, that are in practice till now, are invasive and come at a considerable risk. They often require the use of general anesthesia, bowel resection as well as abdominal incisions.

Therefore it is a matter of concern and there is a need to address aforesaid problems.

### SUMMARY OF THE INVENTION

The following description is a complete disclosure and therefore lays down the aspects of the proposed solution. A person skilled in the art would appreciate that this complete specification may comprise one or more elements and sub-elements thereof, including but not limited to, routines, methods, processes, operations, systems, apparatuses and devices, whether named or unnamed, without deviating from the scope of this complete application. Those skilled in the art would also appreciate that the invention disclosed in this complete application may be carried out by methods and/or through systems which are equivalent to the solution disclosed in this complete application, and therefore any definition/description of the said equivalents in the complete specification should be construed as being amply covered in the complete specification.

The present invention is intended to address at least one of the abovementioned problems and/or disadvantages and to provide a suitable solution. Accordingly, an aspect of the present invention is to provide an external endoluminal fixator device to fix rectal prolapse and/or vaginal and/or uterine and/or urinary bladder prolapse by passing through rectum and vagina respectively.

In accordance with an aspect of the present invention, a description of an external endoluminal fixator device to fix rectal prolapse and/or vaginal and/or uterine and/or urinary bladder prolapse by passing through rectum and vagina respectively is provided. The device includes a head configured at distal end of the external endoluminal fixator device such that the head passes through an anorectum or a vagina of a human body. In an embodiment, the head is made of transparent silicone gel so as to allow light of an endovision system to pass-through, creating trans-illumination in the anterior abdominal wall. The device then includes a hollow shaft connected with the head in an angulation corresponding to usage in the anorectum or a sacral hollow or the vagina of the body, so as to enable entry of one or more endoluminal instruments. In an embodiment, the hollow shaft is configured with an endoluminal forceps or a grasper to hold one or more percutaneous suture needles introduced through the anterior abdominal wall from outside or into the anterior abdominal wall form inside. The device further includes an endoluminal fixator device handle connected with the hollow shaft at proximal end of the external endoluminal fixator device, configured to remain outside of anus or vagina to have a sturdy grip so as to push up the head of the endoluminal fixator device placed either trans-anally or vaginally so as to push the prolapsed anorectum or the vagina against undersurface of the anterior abdominal wall. In an embodiment, the endoluminal fixator device handle is made hollow to allow passage to the one or more endoluminal instruments or an external flexible endoscope.

In accordance with an aspect of the present invention, a method to fix rectal prolapse and/or vaginal and/or uterine and/or urinary bladder prolapse by passing through rectum and vagina respectively with an external endoluminal fixator device is provided. The method includes introducing a head of endoluminal fixator device on an apex of the protruded rectum or vagina. In an embodiment, the head is configured at distal end of the external endoluminal fixator device such that the head passes through the anorectum or a vagina of the human body, said head is made of transparent silicone gel so as to allow light of an endovision system to pass-through, creating trans-illumination in the anterior abdominal wall. The method then includes lifting the rectum or the vagina to the under-surface of the anterior abdominal wall by help of a hollow shaft connected with the head which is made of an opaque translucent biopolymer/metal material (disposable or reusable respectively) with an angulation corresponding to usage in the anorectum or a sacral hollow or the vagina of the body, so as to enable entry of one or more endoluminal instruments through the hollow shaft. In an embodiment, the hollow shaft is configured with an endoluminal forceps or a grasper to hold one or more percutaneous suture needles. The method further includes stitching the rectum or the vagina to the under surface of the anterior abdominal wall by one or more percutaneous suture needles with the help of an endoluminal fixator device handle connected with the hollow shaft at proximal end of the external endoluminal fixator device, configured to remain outside of anus or vagina to have a sturdy grip so as to push up the head of the endoluminal fixator device either trans-anally or vaginally with the anorectum or the vagina against undersurface of the anterior abdominal wall. In an embodiment, the endoluminal fixator device handle is made hollow to allow passage to the one or more endoluminal instruments or a flexible endoscope.

Other aspects, advantages and salient features of the invention will become apparent to those skilled in the art from the following detailed description, which, taken in conjunction with the annexed drawings, discloses exemplary embodiments of the invention.

### BRIEF DESCRIPTION OF DRAWINGS

The above and other aspects, features and advantages of certain exemplary embodiments of the present invention will be more apparent from the following detailed description taken in conjunction with the accompanying drawings, in which:

FIG. 1 illustrates a high level block diagram of a preferred embodiment of the claimed system,

FIG. 2 illustrates a high level flow chart of a preferred embodiment of the claimed process, and

FIG. 3 illustrates is an exemplary example of the present invention where an external endoluminal fixator device is used to fix rectal/vaginal/uterine prolapse.

Persons skilled in the art will appreciate that elements in the figures are illustrated for simplicity and clarity and may have not been drawn to scale. For example, the dimensions of some of the elements in the figure may be exaggerated relative to other elements to help to improve understanding of various exemplary embodiments of the present disclosure.

### DETAILED DESCRIPTION

The following description with reference to the accompanying drawings is provided to assist in a comprehensive understanding of exemplary embodiments of the invention as defined by the claims and their equivalents. It includes various specific details to assist in that understanding but these are to be regarded as merely exemplary. Accordingly, those of ordinary skill in the art will recognize that various changes and modifications of the embodiments described herein can be made without departing from the scope and spirit of the invention. In addition, descriptions of well-known functions and constructions are omitted for clarity and conciseness.

The terms and words used in the following description and claims are not limited to the bibliographical meanings, but, are merely used by the inventor to enable a clear and consistent understanding of the invention. Accordingly, it should be apparent to those skilled in the art that the following description of exemplary embodiments of the present invention are provided for illustration purpose only and not for the purpose of limiting the invention as defined by the appended claims and their equivalents.

It is to be understood that the singular forms “a” “an” and “the” include plural referents unless the context clearly dictates otherwise. Thus, for example, reference to “a component surface” includes reference to one or more of such surfaces.

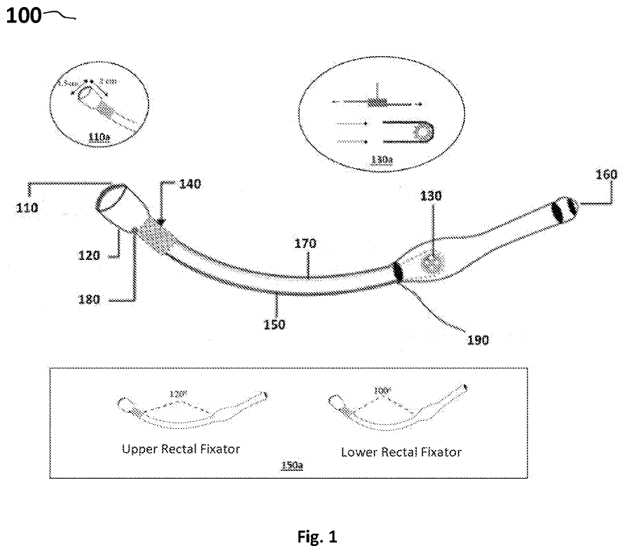

FIG. 1 to FIG. 3, discussed below, is non-limiting illustrations which may be used to explain and describe the invention used to explain and describe the invention disclosed herein. Persons skilled in the art will appreciate that the purpose of these figures is to provide clarity on the concepts associated with the various technical embodiments of this invention. These figures may include block diagrams and/or flowcharts and/or other simplistic representations and may not always indicate the real time operations taking place in the invention.

Accordingly, FIG. 1 illustrates a high level block diagram of a preferred embodiment of the claimed system 100. In accordance with an aspect of the present invention, an external endoluminal fixator device 100 to fix rectal prolapse and/or vaginal and/or uterine and/or urinary bladder prolapse by passing through rectum and vagina respectively is provided. The device includes a head 110 configured at distal end of the external endoluminal fixator device 100 such that the head passes through an anorectum or a vagina of the human body. In an embodiment, the head is made of transparent silicone gel 120 so as to allow light of an endovision system to pass-through, creating trans-illumination in an anterior abdominal wall.

In an embodiment, the head 110 can be, for a non-limiting example, of dimension 1.5 cm×2 cm for an endorectal fixator as shown in 110a and 1 cm×1.5 cm for endovaginal fixator. In a preferred embodiment, the head 110 is made of transparent silicone gel 120 that acts as a receptacle for passage of one or more percutaneous suture needles.

In one other embodiment, the silicone gel is configured with a thin rim so as to allow the one or more percutaneous suture needles to pass through a lumen of the endoluminal fixator device placed inside the rectum or the vagina to outside of the anterior abdominal wall from inside. Transparent silicone gel 120 is selected so as to allow light of, for a non-limiting example, an external flexible endoscope and/or inbuilt endovision system to pass through the transparent silicone gel 120 to create a trans-illumination in the anterior abdominal wall.

In a preferred embodiment, an interior wall of the head 110 is configured with magnetic metallic lining to attract and pull the one or more percutaneous suture needles in case the one or more percutaneous suture needles are introduced through abdominal wall from outside to the head to the endoluminal fixator device placed intraluminally either inside the rectum or the vagina.

In yet another embodiment the interior wall of the head 110 is configured with non-magnetic lining in case the one or more percutaneous suture needles are introduced through the endoluminal fixator device handle through the hollow shaft by endoluminal instruments, across the thin rim of the silicone gel head and then into the abdominal wall that is to say from inside to the outside.

The head as claimed in claim 1, wherein, the head is configured with a de-attachable wheel 130 connected at a bending section 140 of the head using a pulley 130a, so that if attached can enable the head to move up and down, or else the head remains fixed at a position.

The device then includes a hollow shaft 150 connected with the head 110 which is in an angulation corresponding to usage in the anorectum or a sacral hollow or the vagina of the body, so as to enable entry of one or more endoluminal instruments. In an embodiment, the hollow shaft 150 is configured with a forceps or a grasper to hold one or more percutaneous suture needles.

In an non-limiting example, the hollow shaft 150 is made of either an opaque/translucent biopolymer so as to make the hollow shaft biodegradable/disposable or opaque metallic steel to make the hollow shaft reusable and is hollow with a gentle angulation 150a corresponding to that of the anorectum/sacral hollow and vagina. Diameter of cross-section hollow is about 1 cm inside, allowing entry of a 9 mm flexible endoscope for the endoluminal fixator device 100 and around 6 mm for endovaginal/cervical fixator allowing entry of a 5 mm choledochoscope/bronchoscope. Average length for endoluminal fixator device 100 is about 20 cm for the endorectal fixator and 10 cm for the endovaginal/cervical fixator. The endoluminal fixator device 100 has a shaft angulation of 120 degree to fix the upper rectum (upper endorectal fixator) and 100 degree for the lower rectum (lower rectal fixator). The endovaginal/cervical fixator curve is about 100 degree.

In yet another embodiment, the device further includes an endoluminal fixator device handle 160 connected with the hollow shaft 150 at proximal end of the external endoluminal fixator device, configured to remain outside of anus or vagina to have a sturdy grip so as to push up the head of the endoluminal fixator device either trans-anally or vaginally with the anorectum or the vagina against undersurface of the anterior abdominal wall. In an embodiment, the endoluminal fixator device handle 160 is made hollow to allow passage to the one or more endoluminal instruments or an external flexible endoscope.

In yet another preferred embodiment, the external endoluminal fixator device 100 includes an endovision channel 170 having a CCD camera chip 180 at a base of the head configured with the walls of the hollow shaft 150 through an optical cable configured with the hollow shaft 150 or through a CMOS chip for wireless transmission of intraluminal images so as to achieve an independent endovision system without a requirement of insertion of an external flexible endoscope for vision.

In an embodiment, the optical cable is configured with a CCD camera as distal end for vision and a user interface at proximal end 190 of the optical cable for viewing or monitoring so as to get more space inside lumen of the hollow shaft 150 to introduce one or more instrument for intervention.

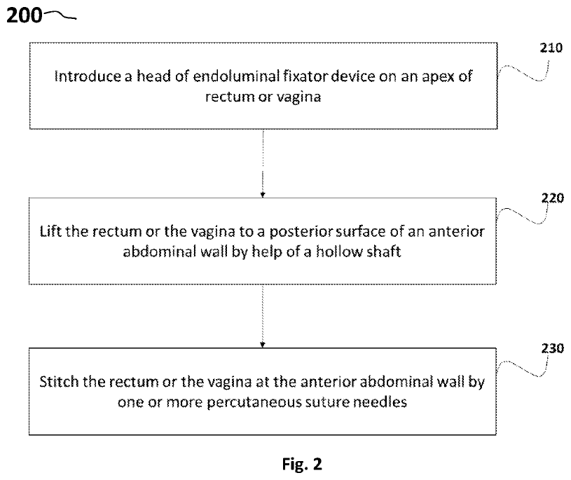

Accordingly, FIG. 2 FIG. 2 illustrates a high level flow chart of a preferred embodiment of the claimed process. In accordance with an aspect of the present invention, a method 200 to fix rectal prolapse and/or vaginal and/or uterine and/or urinary bladder prolapse by passing through rectum and vagina respectively with an external endoluminal fixator device is provided. At STEP 210 a head of endoluminal fixator device is introduced at the apex of the rectum or vagina. In an embodiment, the head is configured at distal end of the external endoluminal fixator device such that the head passes through an anorectum or a vagina of a body, said head is made of transparent silicone gel so as to allow light of an endovision system to pass-through, creating trans-illumination in an anterior abdominal wall.

In a preferred embodiment, the one or more percutaneous suture needles is attracted by magnetic metallic lining as the metallic line creates magnetic effect to attract and pull the one or more percutaneous suture needles, inserted through abdominal wall to inside the silicone gel head of the endoluminal fixator device, placed within the pushed up rectum or vagina against the undersurface of anterior abdominal wall.

In one other embodiment the head is made flexible with a de-attachable wheel connected at a bending section of the head using a pulley, so that if attached can enable the head to move up and down, else the head remains fixed at a position.

In yet another embodiment, the silicone gel is configured with a thin rim so as to allow the one or more percutaneous suture needles to pass through a lumen of the endoluminal fixator device placed inside the rectum or the vagina to outside (external surface) of the anterior abdominal wall, from within the hollow of the shaft held by endoluminal grasper forcep.

At STEP 220, the rectum or the vagina is lifted against the posterior (under) surface of the anterior abdominal wall with the help of a hollow shaft connected with the head which is made of an opaque/translucent biopolymer material or opaque metallic steel with an angulation corresponding to usage in the anorectum or a sacral hollow or the vagina of the body, so as to enable entry of one or more endoluminal instruments. In an embodiment, the hollow shaft is configured with endoluminal forceps or a grasper to hold one or more percutaneous suture needles.

At STEP 230, the rectum or the vagina is suture fixed at the under surface of anterior abdominal wall by one or more percutaneous suture needles with the help of an endoluminal fixator device handle connected with the hollow shaft at proximal end of the external endoluminal fixator handle, configured to remain outside of anus or vagina to have a sturdy grip so as to push up the head of the endoluminal fixator device either trans-anally or vaginally within the anorectum or the vagina against undersurface of the anterior abdominal wall. In an embodiment, the endoluminal fixator device handle is made hollow to allow passage to the one or more endoluminal instrument or an external flexible endoscope.

In a preferred embodiment a camera chip is fixed at a base of the head so as to create an endovision channel, wherein the camera chip can be connected through a CMOS to make a wireless transmission of intraluminal images so as to keep hollow space of the hollow shaft available for introduction of one or more endoluminal instruments.

In yet another embodiment, the optical cable is configured with a CCD camera at distal end for vision and a user interface at proximal end of the optical cable for viewing or monitoring so as to get more space inside lumen of the hollow shaft to introduce one or more instrument for intervention. The optical cable in this case travels within the walls of the shaft of the endoluminal fixator.

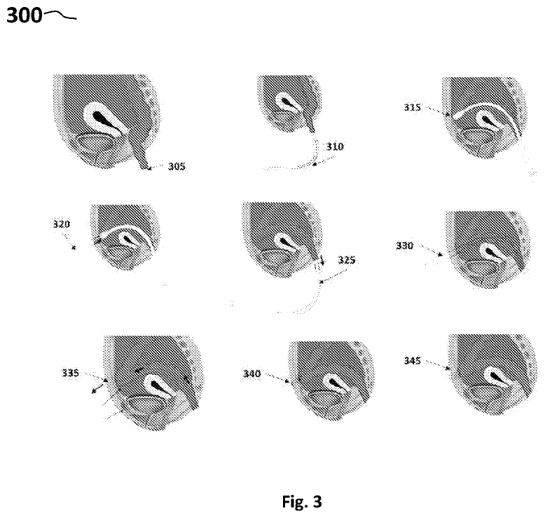

FIG. 3 illustrates is an exemplary example of the present invention where an external endoluminal fixator device is used to fix rectal prolapse.

A rectum prolapse 305 is intended to treat; the external endoluminal fixator device is placed at apex of prolapse 310. Upper rectum is lifted by the external endoluminal fixator device against the under (posterior) surface of anterior abdominal wall 315. One or more percutaneous suture needles are introduced 320 through abdominal wall into the endoluminal fixator device, placed inside the lumen of the reduced Rectum.

The endoluminal fixator device is pulled out 330 with the percutaneous suture needles carrying suture and is knotted outside the anal canal/vagina 335. The external ends of the two suture needs are pulled at the anterior abdominal wall pulling the externally tied knot inside anorectal/vaginal lumen along with the anterior wall of prolapsed rectum/vagina against the undersurface of the anterior abdominal wall 340. The external ends of the suture needle are then tied at the anterior abdominal wall fixing the anterior rectal/vaginal wall onto the undersurface of anterior abdominal wall. In an embodiment; multiple sutures can be placed by repeating the aforesaid process so as to ensure a good anterior (ventral) abdominal wall rectopexy or vagino-cervicopexy S345.

While the invention has been shown and described with reference to certain exemplary embodiments thereof, it will be understood by those skilled in the art that various changes in form and details may be made therein without departing from the spirit and scope of the invention as defined by the appended claims and their equivalents.

In relation to the preceding specification, it is reiterated that the present disclosure and its advantages have been described with reference to exemplary embodiments and that, a person of ordinary skill in the art would appreciate that various modifications and changes may be made, without departing from the scope of the present disclosure, as set forth in the appended claims and their equivalents. Furthermore, it is re-emphasized that the preceding specification and figures are to be regarded as illustrative examples of the present disclosure, rather than in restrictive sense. All such possible modifications are intended to be included within the scope of the present invention.

I/We claim:

1. An external endoluminal fixator device to fix rectal prolapse and/or vaginal and/or uterine and/or urinary bladder prolapse by passing intraluminally through rectum and vagina respectively, wherein the external endoluminal fixating device is characterized by:
a head configured at distal end of the external endoluminal fixator device such that the head passes through an anorectum or a vagina of a human body, wherein the head is made of transparent silicone gel so as to allow light of an endovision system to pass-through, creating trans-illumination in an anterior abdominal wall;
a hollow shaft connected with the head which is in an angulation corresponding to usage in the anorectum or a sacral hollow or the vagina of the human body, so as to enable entry of one or more endoluminal instruments, wherein the hollow shaft is configured with an endoluminal forceps or a grasper to hold one or more percutaneous suture needles; or allow passage of an external flexible endoscope and
an endoluminal fixator device handle connected with the hollow shaft at proximal end of the external endoluminal fixator device, configured to remain outside anus or vagina to have a sturdy grip so as to push up the head of the endoluminal fixator device placed either trans-anally or vaginally so as to push the prolapsed anorectum or the vagina and/or cervix up and against undersurface of the anterior abdominal wall, wherein, the endoluminal fixator device handle is made hollow to allow passage to the one or more endoluminal instruments or an external flexible endoscope.

2. The head as claimed in claim 1, wherein the interior wall of the head is configured with magnetic metallic lining to attract and pull one or more percutaneous suture needles which are introduced from outside through the anterior abdominal wall into the head of the endoluminal fixator device placed inside the reduced rectum or vagina/cervix pushing them up and against the undersurface of anterior abdominal wall, and is configured with non-magnetic-metallic lining in case one or more percutaneous suture needles are introduced through the hollow of endoluminal fixator device handle/shaft/to its head and then through the anterior walls of either Rectum or vagina/cervix into the anterior abdominal wall, by endoluminal instruments passed through the hollow of the shaft.

3. The head as claimed in claim 1, wherein, the head is configured with a de-attachable wheel connected at a bending section of the head/shaft using a pulley, so that if attached can enable the head to move up and down, or else the head remains fixed at a position.

4. The external endoluminal fixator device as claimed in claim 1, wherein the system comprises an endovision channel having a CMOS camera chip at a base of the head for wireless transmission of intraluminal images so as to achieve an independent endovision system without a requirement of insertion of an external flexible endoscope and also to get more space inside lumen of the hollow shaft to introduce one or more instruments for intervention.

5. The optical cable as claimed in claim 4, wherein the optical cable is configured with a CCD camera as distal end for vision and a user interface at proximal end of the optical cable for viewing or monitoring so as to get more space inside lumen of the hollow shaft to introduce one or more instruments for intervention.

6. The head as claimed in claim 1, wherein the silicone gel is configured with a thin rim so as to allow the one or more percutaneous suture needles to pass through a lumen of the endoluminal fixator device placed inside the rectum or the vagina to outside of the anterior abdominal wall from inside.

7. The hollow shaft as claimed in claim 1, wherein the hollow shaft can be made either of an opaque or translucent biopolymer so as to make the hollow shaft disposable or of stainless steel so as to make the hollow shaft reusable.

8. A method to fix rectal prolapse and vaginal prolapse by passing through rectum and/or vaginal and/or uterine and/or urinary bladder respectively with an external endoluminal fixator device, wherein the method comprises steps of:
introducing a head of endoluminal fixator device at the apex of the rectum or vagina, wherein the head is configured at distal end of the external endoluminal fixator device such that the head passes through the anorectum or a vagina of the body, said head is made of transparent silicone gel so as to allow light of an endovision system to pass-through, creating trans-illumination in an anterior abdominal wall;
lifting and pushing the prolapsed rectum or the/cervixto an under (posterior) surface of the anterior abdominal wall by help of a hollow shaft connected in an angulation corresponding to usage in the anorectum or a sacral hollow or the vagina of the body, so as to enable entry of one or more endoluminal instruments, wherein the hollow shaft is configured with endoluminal forceps or a grasper to hold one or more percutaneous suture needles; and
fixing the rectum or the vagina/uterine cervixto the undersurface of the anterior abdominal wall by one or more percutaneous suture needles with the help of an endoluminal fixator device handle connected with the hollow shaft at proximal end of the external endoluminal fixator device, configured to remain outside of anus or vagina to have a sturdy grip so as to push up the head of the endoluminal fixator device either trans-anally or vaginally with the anorectum or the vagina and/or cervix against undersurface of the anterior abdominal wall, wherein, the endoluminal fixator device handle is made hollow to allow passage to the one or more endoluminal instruments or a flexible endoscope for independent vision

9. The method as claimed in claim 8, wherein the method comprises steps of attracting and pulling one or more percutaneous suture needles by magnetic metallic lining so as to create a magnetic effect while the one or more percutaneous suture needles are inserted through abdominal wall into the silicone gel head to the endoluminal fixator device, placed inside the lumen of the reduced Rectum or vagina/cervix

10. The method as claimed in claim 8, wherein, the method comprises steps of making the head flexible with a de-attachable wheel connected at a bending section of the head-shaft using a pulley, so that if attached can enable the head to move up and down, else the head remains fixed at a position.

11. The method as claimed in claim 8, wherein the method comprises steps of fixing a camera chip at a base of the head so as to create an endovision channel, wherein the camera chip can be CMOS to make a wireless transmission of intraluminal images or connected with an optical cable configures with the walls of the hollow shaft so as to keep hollow space of the hollow shaft available for introduction of one or more endoluminal instruments.

12. The method as claimed in claim 11, wherein the method comprises steps of configuring the optical cable with a CCD camera at distal end for vision and a user interface at proximal end of the optical cable for viewing or monitoring so as to get more space inside lumen of the hollow shaft to introduce one or more instrument for intervention.

13. The method as claimed in claim 8, wherein the method comprises steps of configuring the silicone gel with a thin rim so as to allow the one or more percutaneous suture needles to pass through a lumen of the endoluminal fixator device placed inside the rectum or the vagina to outside of the anterior abdominal wall.

14. The method as claimed in claim 8, wherein the hollow shaft can be made either of an opaque or translucent biopolymer so as to make the hollow shaft disposable or of stainless steel so as to make the hollow shaft reusable.
